(19) United States
(12) Patent Application Publication
(10) Pub. No.: US 2022/0055972 A1
(43) Pub. Date: Feb. 24, 2022

(54) ALKANE OXIDATIVE DEHYDROGENATION AND/OR ALKENE OXIDATION

(71) Applicant: SHELL OIL COMPANY, HOUSTON, TX (US)

(72) Inventors: Ronald Jan SCHOONEBEEK, Amsterdam (NL); Guus VAN ROSSUM, Amsterdam (NL)

(21) Appl. No.: 17/311,535

(22) PCT Filed: Dec. 6, 2019
(86) PCT No.: PCT/EP2019/083960

§ 371 Date: Jun. 7, 2021

(30) Foreign Application Priority Data
Dec. 11, 2018 (EP) 18211590.7

(51) Int. Cl. C07C 5/48; B01J 23/00

(52) U.S. Cl. CPC C07C 5/48; B01J 23/002; C07C 2523/22; C07C 2523/28; B01J 2523/68; C07C 2523/20; B01J 2523/00; B01J 2523/55; B01J 2523/64; C07C 2527/057

(57) ABSTRACT

The invention relates to a process of the oxidative dehydrogenation of an alkane containing 2 to 6 carbon atoms and/or the oxidation of an alkene containing 2 to 6 carbon atoms, wherein the alkane and/or alkene is contacted with oxygen in the presence of a catalyst comprising a mixed metal oxide and one or more diluents selected from the group consisting of carbon dioxide, carbon monoxide and steam, and wherein the conversion of the alkane and/or alkene is at least 40%.

### FIELD OF THE INVENTION

The present invention relates to a process for alkane oxidative dehydrogenation and/or alkene oxidation.

### BACKGROUND OF THE INVENTION

It is known to oxidatively dehydrogenate alkanes, such as alkanes containing 2 to 6 carbon atoms, for example ethane or propane resulting in ethylene and propylene, respectively, in an oxidative dehydrogenation (oxydehydrogenation; ODH) process. Examples of alkane ODH processes, including catalysts and other process conditions, are for example disclosed in U.S. Pat. No. 7,091,377, WO2003064035, US20040147393, WO2010096909 and US20100256432. Mixed metal oxide catalysts containing molybdenum (Mo), vanadium (V), niobium (Nb) and optionally tellurium (Te) as the metals, can be used as such oxydehydrogenation catalysts. Such catalysts may also be used in the direct oxidation of alkenes to carboxylic acids, such as in the oxidation of alkenes containing 2 to 6 carbon atoms, for example ethylene or propylene resulting in acetic acid and acrylic acid, respectively.

US20160326070 disclose a process for oxydehydrogenating an alkane to a corresponding alkene comprising: providing a feed of at least an alkane and oxygen as oxidizing agent to a reactor; converting the alkane to a product stream which includes the corresponding alkene by oxydehydrogenation of the alkane with oxygen in the reactor in the presence of a catalyst, wherein the feed further includes a diluent comprising CO₂ as an oxidizing agent. Carbon dioxide (CO₂) is not only a diluent but may also act as a reactant in the following reaction: CO₂+ethane→CO+H₂O+ethylene.

Further, above-mentioned US20160326070 discloses the operation of an ethane recycle in a ratio of recycle ethane to fresh ethane of from 1:1 to 4:1, wherein “fresh ethane” is ethane at the time of its first injection into the reactor and “recycle ethane” is unconverted ethane being reinjected. By recycling unconverted ethane, the overall conversion is improved. This is different from the conversion per pass. An “overall conversion” of a compound “A” is generally defined as (moles of A reacted overall)/(mole of fresh feed), whereas a “conversion per pass” is defined as (moles of A reacted in a single pass)/(mole of A fed to the reactor). Usually, a relatively high ratio of recycle unconverted reactant to fresh reactant is needed to keep the conversion per pass low to safeguard a certain desired selectivity.

It is an object of the present invention to provide a process for alkane oxidative dehydrogenation and/or alkene oxidation wherein there is less unconverted alkane and/or alkene that needs to be recycled to the reactor, while preferably at the same time maintaining the selectivity at a relatively high level. In addition, it is an object of the present invention to provide a process for alkane oxidative dehydrogenation and/or alkene oxidation wherein at a certain conversion a relatively high selectivity is obtained.

### SUMMARY OF THE INVENTION

Surprisingly it was found that one or more of the above-mentioned objects may be achieved by contacting an alkane and/or alkene with oxygen in the presence of a catalyst comprising a mixed metal oxide and one or more diluents selected from the group consisting of carbon dioxide, carbon monoxide and steam, wherein the conversion of the alkane and/or alkene is at least 40%.

Accordingly, the present invention relates to a process of the oxidative dehydrogenation of an alkane containing 2 to 6 carbon atoms and/or the oxidation of an alkene containing 2 to 6 carbon atoms, wherein the alkane and/or alkene is contacted with oxygen in the presence of a catalyst comprising a mixed metal oxide and one or more diluents selected from the group consisting of carbon dioxide, carbon monoxide and steam, and wherein the conversion of the alkane and/or alkene is at least 40%.

### DETAILED DESCRIPTION OF THE INVENTION

While the process of the present invention and a composition or stream used in said process are described in terms of “comprising”, “containing” or “including” one or more various described steps and components, they can also “consist essentially of” or “consist of” said one or more various described steps and components.

In the context of the present invention, in a case where a composition or stream comprises two or more components, these components are to be selected in an overall amount not to exceed 100%.

Within the present specification, “substantially no” means that no detectible amount of the component in question is present in the composition or stream.

Further, where upper and lower limits are quoted for a property then a range of values defined by a combination of any of the upper limits with any of the lower limits is also implied.

Further, within the present specification, by “fresh alkane” reference is made to alkane which does not comprise unconverted alkane. Within the present specification, by “unconverted alkane” reference is made to alkane that was subjected to the process of the present invention for the first time, but which was not converted. Similar definitions for “fresh alkene” and “unconverted alkene” apply.

In the present alkane oxidative dehydrogenation and/or alkene oxidation process, an alkane containing 2 to 6 carbon atoms (hereinafter the “alkane”) and/or the oxidation of an alkene containing 2 to 6 carbon atoms (hereinafter the “alkene”) is contacted with oxygen in the presence of a catalyst comprising a mixed metal oxide and one or more diluents selected from the group consisting of carbon dioxide, carbon monoxide and steam.

Further, in the present invention the conversion of the alkane and/or alkene is at least 40%. By said conversion, reference is made to a “conversion per pass”, which is defined as (moles of alkane and/or alkene reacted in a single pass)/(mole of alkane and/or alkene fed to the reactor). Such conversion per pass can be controlled by varying one or more parameters. Such parameters include temperature, pressure, nature of the catalyst, amount of the catalyst and amount of oxygen.

In the present invention, the conversion per pass of the alkane and/or alkene is controlled to be at least 40%. It has appeared that at such relatively high conversion per pass, the selectivity to the desired product(s) may still be relatively high in the presence of a diluent selected from the group consisting of carbon dioxide, carbon monoxide and steam. Thus, advantageously, in the present invention wherein such diluent is used, the conversion per pass can be increased thereby reducing the volume of unreacted alkane and/or alkene to be recycled, while at the same time achieving a relatively high selectivity to the desired product(s) thereby resulting in a smaller amount of undesired products and thus increasing the efficiency of the overall process. Thus, conversely, in the present invention a relatively high selectivity at a certain (i.e. same) conversion may be obtained. In the present invention, in a case where ethane is the freshly fed reactant the desired products comprise ethylene and acetic acid, whereas in a case of ethylene as freshly fed reactant the desired product comprises acetic acid.

Preferably, in the present invention, the conversion per pass of the alkane and/or alkene is controlled to be at least 45%, more preferably at least 50%, more preferably at least 55%, more preferably at least 60%, more preferably at least 65%, most preferably at least 70%. Further, preferably, said conversion per pass is at most 99%, more preferably at most 95%, more preferably at most 90%, more preferably at most 85%, more preferably at most 80%, more preferably at most 75%, more preferably at most 70%, more preferably at most 65%, most preferably at most 60%.

Preferably, in the present invention, the alkane containing 2 to 6 carbon atoms is a linear alkane in which case said alkane may be selected from the group consisting of ethane, propane, butane, pentane and hexane. Further, preferably, said alkane contains 2 to 4 carbon atoms and is selected from the group consisting of ethane, propane and butane. More preferably, said alkane is ethane or propane. Most preferably, said alkane is ethane. In case an alkane is used, the present invention is referred to as an alkane oxidative dehydrogenation process.

Further, preferably, in the present invention, the alkene containing 2 to 6 carbon atoms is a linear alkene in which case said alkene may be selected from the group consisting of ethylene, propylene, butene, pentene and hexene. Further, preferably, said alkene contains 2 to 4 carbon atoms and is selected from the group consisting of ethylene, propylene and butene. More preferably, said alkene is ethylene or propylene. In case an alkene is used, the present invention is referred to as an alkene oxidation process.

The product of said alkane oxidative dehydrogenation process may comprise the dehydrogenated equivalent of the alkane, that is to say the corresponding alkene. For example, in the case of ethane such product may comprise ethylene, in the case of propane such product may comprise propylene, and so on. Such dehydrogenated equivalent of the alkane is initially formed in said alkane oxidative dehydrogenation process. However, in said same process, said dehydrogenated equivalent may be further oxidized under the same conditions into the corresponding carboxylic acid which may or may not contain one or more unsaturated double carbon-carbon bonds. As mentioned above, it is preferred that the alkane containing 2 to 6 carbon atoms is ethane or propane. In the case of ethane, the product of said alkane oxidative dehydrogenation process may comprise ethylene and/or acetic acid, preferably ethylene. Further, in the case of propane, the product of said alkane oxidative dehydrogenation process may comprise propylene and/or acrylic acid, preferably acrylic acid.

The product of said alkene oxidation process comprises the oxidized equivalent of the alkene. Preferably, said oxidized equivalent of the alkene is the corresponding carboxylic acid. Said carboxylic acid may or may not contain one or more unsaturated double carbon-carbon bonds. As mentioned above, it is preferred that the alkene containing 2 to 6 carbon atoms is ethylene or propylene. In the case of ethylene, the product of said alkene oxidation process may comprise acetic acid. Further, in the case of propylene, the product of said alkene oxidation process may comprise acrylic acid.

In the present invention, the alkane and/or alkene, oxygen (O₂) and the one or more diluents may be fed to a reactor. Said components may be fed to the reactor together or separately. That is to say, one or more feed streams, suitably gas streams, comprising one or more of said components may be fed to the reactor. For example, one feed stream comprising oxygen, the alkane and/or alkene and diluent may be fed to the reactor. Alternatively, two or more feed streams, suitably gas streams, may be fed to the reactor, which feed streams may form a combined stream inside the reactor. For example, one feed stream comprising oxygen, another feed stream comprising the alkane and/or alkene and still another feed stream comprising diluent may be fed to the reactor separately. In the present invention, the alkane and/or alkene, oxygen and diluent are suitably fed to a reactor in the gas phase.

Preferably, in the present invention, that is to say during contacting the alkane and/or alkene with oxygen in the presence of the catalyst, the temperature is of from 300 to 500° C. More preferably, said temperature is of from 310 to 450° C., more preferably of from 320 to 420° C., most preferably of from 330 to 420° C.

Still further, in the present invention, that is to say during contacting the alkane and/or alkene with oxygen in the presence of the catalyst, typical pressures are 0.1-30 or 0.1-20 bara (i.e. “bar absolute”). Further, preferably, said pressure is of from 0.1 to 15 bara, more preferably of from 1 to 12 bara, most preferably of from 2 to 12 bara. Said pressure refers to total pressure.

In the present invention, a diluent is used. The diluent comprises one or more diluents selected from the group consisting of carbon dioxide (CO₂), carbon monoxide (CO) and steam (H₂O). Most preferably, the diluent comprises carbon dioxide. The diluent may comprise carbon dioxide and optionally one or more diluents selected from the group consisting of methane, nitrogen, carbon monoxide and steam, preferably steam and/or nitrogen. Further, the diluent may comprise carbon monoxide and optionally one or more diluents selected from the group consisting of carbon dioxide, methane, nitrogen and steam, preferably steam and/or nitrogen.

Thus, in addition to oxygen and the alkane and/or alkene, diluent is also fed to the present process. In case carbon dioxide is fed to the present process as a diluent, one or more additional diluents, selected from the group consisting of the noble gases, nitrogen, steam and methane, suitably nitrogen and methane, may be fed to the present process. However, in case in the present process carbon dioxide is already fed as a diluent to the present process, there is no need to add any additional diluent. Therefore, suitably, no additional diluent, in particular no steam, is fed to the present process in case carbon dioxide is fed to the present process as a diluent. Some methane may be fed to the present process as an impurity in the C₂₋₆ alkane feed to the present process. Further, some nitrogen may be fed to the present process as an impurity in the oxygen feed to the present process. Upon recycling, these methane and/or nitrogen impurities may accumulate. In these cases, methane and nitrogen function as (additional) diluent. Preferably, a diluent consisting of carbon dioxide is used in the present invention.

Generally, the proportion of the overall feed stream to the present process which is attributable to a diluent is in the range from 5 to 90 vol. %, preferably from 25 to 75 vol. %. Said proportion may be at least 5 vol. % or at least 10 vol. % or at least 15 vol. % or at least 20 vol. % or at least 25 vol. % and may be at most 90 vol. % or at most 80 vol. % or at most 70 vol. % or at most 60 vol. % or at most 50 vol. % or at most 45 vol. % or at most 40 vol. % or at most 35 vol. %.

Preferably, in the case of an isothermally operated reactor, the proportion of the overall feed stream to the present process which is attributable to a diluent is in the range from 5 to 90 vol. %, preferably from 25 to 75 vol. % and more preferably from 40 to 60 vol. %. Further, preferably, in the case of an adiabatically operated reactor, the proportion of the overall feed stream to the present process which is attributable to a diluent is in the range from 50 to 95 vol. %, preferably from 60 to 90 vol. % and more preferably from 70 to 85 vol. %.

Preferably, the diluent as fed to the present process comprises from 1 to 100 vol. %, more preferably 5 to 100 vol. %, more preferably 10 to 100 vol. %, more preferably 20 to 100 vol. %, more preferably 40 to 100 vol. %, more preferably 60 to 100 vol. %, more preferably 80 to 100 vol. %, more preferably 90 to 100 vol. %, more preferably 95 to 100 vol. %, and most preferably 99 to 100 vol. % of carbon dioxide, the balance consisting of one or more other diluents, selected from the group consisting of the noble gases, nitrogen, steam and methane. Diluents other than carbon dioxide may be used in any desired ratio relative to each other. When one or more of said additional diluents other than carbon dioxide are fed to the present process, the upper limit for the proportion of carbon dioxide in the diluent may be 20 vol. %, preferably 40 vol. %, more preferably 60 vol. %, more preferably 80 vol. %, more preferably 90 vol. %, more preferably 95 vol. %, and most preferably 99 vol. %.

The oxygen as fed to the present process is an oxidizing agent, thereby resulting in oxidative dehydrogenation (ODH) of the alkane or oxidation of the alkene. Said oxygen may originate from any source, such as for example air. Suitable ranges for the molar ratio of oxygen to the alkane and/or alkene cover ratios below, at and above the stoichiometric molar ratio (which is 0.5 for the ethane ODH reaction), suitably of from 0.01 to 1.1, more suitably of from 0.01 to 1, more suitably of from 0.05 to 0.8, most suitably of from 0.05 to 0.7. In one embodiment, the molar ratio of oxygen to the alkane and/or alkene is of from 0.05 to 0.5, more suitably of from 0.05 to 0.47, most suitably of from 0.1 to 0.45. Further, in another embodiment, the molar ratio of oxygen to the alkane and/or alkene is of from 0.5 to 1.1, more suitably of from 0.53 to 1, most suitably of from 0.55 to 0.9. Said ratio of oxygen to alkane and/or alkene is the ratio before oxygen and the alkane and/or alkene are contacted with the catalyst. In other words, said ratio of oxygen to the alkane and/or alkene is the ratio of oxygen as fed to the alkane and/or alkene as fed. Obviously, after contact with the catalyst, at least part of the oxygen and the alkane and/or alkene gets consumed. Further, said “alkane and/or alkene” in said molar ratio of oxygen to the alkane and/or alkene comprises both fresh alkane and/or alkene and recycled (unconverted) alkane and/or alkene.

Preferably, pure or substantially pure oxygen (O₂) is used as oxidizing agent in the process of the present invention. Within the present specification, by “pure or substantially pure oxygen” reference is made to oxygen that may contain a relatively small amount of one or more contaminants, including for example nitrogen (N₂), which latter amount may be at most 1 vol. %, suitably at most 7,000 parts per million by volume (ppmv), more suitably at most 5,000 ppmv, more suitably at most 3,000 ppmv, more suitably at most 1,000 ppmv, more suitably at most 500 ppmv, more suitably at most 300 ppmv, more suitably at most 200 ppmv, more suitably at most 100 ppmv, more suitably at most 50 ppmv, more suitably at most 30 ppmv, most suitably at most 10 ppmv.

Alternatively, however, it is also possible to use air or oxygen-enriched air as oxidizing agent in the present process. Such air or oxygen-enriched air would still comprise nitrogen (N₂), in an amount exceeding 1 vol. % up to 78 vol. % (air), suitably of from 1 to 50% vol. %, more suitably 1 to 30 vol. %, more suitably 1 to 20 vol. %, more suitably 1 to 10 vol. %, most suitably 1 to 5 vol. %. Said nitrogen would function as (additional) diluent.

In the present process, the catalyst is a catalyst comprising a mixed metal oxide. Preferably, the catalyst is a heterogeneous catalyst. Further, preferably, the catalyst is a mixed metal oxide catalyst containing molybdenum, vanadium, niobium and optionally tellurium as the metals, which catalyst may have the following formula:

Mo₁VₐTe_{b}Nb_{c}Oₙ

wherein:

a, b, c and n represent the ratio of the molar amount of the element in question to the molar amount of molybdenum (Mo);

a (for V) is from 0.01 to 1, preferably 0.05 to 0.60, more preferably 0.10 to 0.40, more preferably 0.20 to 0.35, most preferably 0.25 to 0.30;

b (for Te) is 0 or from >0 to 1, preferably 0.01 to 0.40, more preferably 0.05 to 0.30, more preferably 0.05 to 0.20, most preferably 0.09 to 0.15;

c (for Nb) is from >0 to 1, preferably 0.01 to 0.40, more preferably 0.05 to 0.30, more preferably 0.10 to 0.25, most preferably 0.14 to 0.20; and

n (for 0) is a number which is determined by the valency and frequency of elements other than oxygen.

The amount of the catalyst in the present invention is not essential. Preferably, a catalytically effective amount of the catalyst is used, that is to say an amount sufficient to promote the reaction.

The ODH reactor that may be used in the present process may be any reactor, including fixed-bed and fluidized-bed reactors. Suitably, the reactor is a fixed-bed reactor.

Examples of oxydehydrogenation processes, including catalysts and process conditions, are for example disclosed in above-mentioned U.S. Pat. No. 7,091,377, WO2003064035, US20040147393, WO2010096909 and US20100256432, the disclosures of which are herein incorporated by reference.

The work-up of the product stream resulting from the present process may be carried out in any known way. Further, unconverted alkane and/or alkene may be recycled to the present process. Preferably, diluent is also recycled, in particular carbon dioxide. Such work-up and recycle may for example be carried out in a way as disclosed in above-mentioned US20160326070, the disclosure of which is herein incorporated by reference. Further, for example, in the case of methane and/or carbon monoxide in the product stream, such methane and/or carbon monoxide may be separated in a demethanizer as a top stream and then recycled to the present process for use as diluent.

The present invention is further illustrated by the following Examples.

### Examples

(A) Preparation of the Catalyst

A mixed metal oxide catalyst containing molybdenum (Mo), vanadium (V), niobium (Nb) and tellurium (Te) was prepared, for which catalyst the molar ratio of said 4 metals was Mo₁V_{0.29}Nb_{0.17}Te_{0.12}, in the following way.

Two solutions were prepared. Solution 1 was obtained by dissolving 15.8 parts by weight (pbw) of ammonium niobate oxalate and 4 pbw of oxalic acid dihydrate in 160 pbw of water at room temperature. Solution 2 was prepared by dissolving 35.6 pbw of ammonium heptamolybdate tetrahydrate, 6.9 pbw of ammonium metavanadate and 5.8 pbw of telluric acid (Te(OH)₆) in 200 pbw of water at 70° C. 7 pbw of concentrated nitric acid was then added to solution 2.

The 2 solutions were combined, by quickly pouring solution 2 into solution 1 under vigorous stirring, which yielded an orange gel-like precipitate (suspension) having a temperature of about 45° C. This suspension was then aged for about 15 minutes. The suspension was then dried by means of spray drying to remove the water, which yielded a dry, fine powder (the catalyst precursor).

Precipitation and spray drying were executed portion wise at a scale to yield 1 kg of dried material per portion. Said spray drying was carried out by using an air temperature of around 180° C. resulting in a solid temperature of around 80° C.

Subsequently, pre-calcination was carried out in a static ventilated oven wherein the dried catalyst precursor was contacted with air. 250 g portions of catalyst precursor were heated from room temperature to 325° C. at a rate of 100° C./hour and kept at 325° C. for 2 hours and then cooled down. The cooled catalyst precursor was then removed from the oven and further calcined in a nitrogen (N₂) stream in a retort oven. The catalyst precursor was heated from room temperature to 600° C. at a rate of 100° C./hour and kept at 600° C. for 2 hours, after which the catalyst was cooled down to room temperature. The flow of the stream in this calcination step was 150 Nl/hr.

80 pbw of the mixed metal oxide catalyst thus obtained were dry mixed with 17 pwb of cerium oxide (Alfa Aesar Cerium(IV) oxide, Reacton, 99.9%, 5 micron powder).

After dry mixing, a mixture of 0.6 wt. % of Walocel XCS47132, 1 wt. % of Superfloc A-1849RS in water and Bindzil CC301 (a 30 wt. % suspension of silanized silica particles) was slowly added to the solid mixture in an Eirich mixer till the mixture became an extrudable paste. The amount of Bindzil added corresponded to a SiO₂ content of 3 wt. % on dried calcined basis.

After mixing and compacting, the mixture was extruded into trilobe shaped bodies, followed by a final calcination in static air at a temperature of 325° C. for 2 hours.

(B) Catalytic Oxidative Dehydrogenation of Ethane

The catalyst thus prepared was used in experiments involving ethane oxidative dehydrogenation within a pilot plant unit comprising a vertically oriented, cylindrical, stainless steel reactor having an inner diameter of 19 mm. 1.96 kg of the catalyst were loaded in the reactor. The catalyst bed height was 5.6 m.

In the experiments, a gas stream comprising ethane (C₂H₆), oxygen (O₂), methane (CH₄) or carbon dioxide (CO₂), and nitrogen (N₂) was fed to the top of the reactor at a pressure (at the top) of 5 bar, and was sent downwardly through the catalyst bed to the bottom of the reactor. Said gas stream was a combined gas stream comprising a flow of ethane, a flow of oxygen, a flow of methane or carbon dioxide, and a flow of nitrogen. The flow rates of said flows are shown in Table 1 below. The molar ratio of O₂:ethane in this combined inlet gas stream was 0.46:1. The temperature in the reactor was varied to reach a certain ethane conversion, by varying the inlet temperature for a molten salt that was supplied to a shell space of the reactor in a flow pattern that was counter-current with the flow of the feed gas through the reactor. Said salt inlet temperatures (in ° C.) were as follows: Exp. 1=329.4; Exp. 2=337.1; Exp. 3=341.3.

TABLE 1

| | | | | | Total | | |
|---|---|---|---|---|---|---|---|
| Exp. | | CH₄ | CO₂ | N₂ | Diluent | C₂H₆ | O₂ |
| 1 (*) | Nl/hr | 735 | 0 | 150 | 885 | 1462 | 678 |
| | vol. %¹ | 24.3 | | 5.0 | 29.3 | 48.3 | 22.4 |
| | vol. %² | 83.1 | | | | | |
| 2 | Nl/hr | 0 | 750 | 150 | 900 | 1467 | 678 |
| | vol. %¹ | | 24.6 | 4.9 | 29.5 | 48.2 | 22.3 |
| | vol. %² | | 83.3 | | | | |
| 3 | Nl/hr | 0 | 663 | 240 | 903 | 1464 | 678 |
| | vol. %¹ | | 21.8 | 7.9 | 29.7 | 48.1 | 22.3 |
| | vol. %² | | 73.4 | | | | |

(*) = not in accordance with the present invention.
For each experiment, the flow rates in the 1^{st} row are in Nl/hour, wherein “Nl” stands for “normal litre” as measured at standard temperature and pressure, namely 32° F. (0° C.) and 1 bara (100 kPa). The flow rate for “Total Diluent” is the sum of the flow rates for CH₄, CO₂ and N₂.
¹The volume percentages for flow rates in the 2^{nd} row for each experiment are based on the overall feed stream, including all flow rates for CH₄, CO₂, N₂, C₂H₆ and O₂.
²The volume percentage for the flow rate of CH₄ or CO₂ in the 3^{rd} row for each experiment is based on the flow rate for “Total Diluent”.

The conversion of ethane and the product composition were measured with a gas chromatograph (GC) equipped with a thermal conductivity detector (TCD) and with another GC equipped with a flame ionization detector. The water and acetic acid from the reaction were trapped in a quench pot. In Table 2 below, the conversion of ethane and the selectivities towards ethylene and acetic acid for the experiments are shown.

TABLE 2

| Exp. | xC₂H₆ (1) | sC₂H₄ (2) | sAA (3) | s(C₂H₄ + AA) (4) |
|---|---|---|---|---|
| 1 (*) | 51.9 | 84.5 | 8.2 | 92.7 |
| 2 | 51.5 | 87.3 | 8.5 | 95.8 |
| 3 | 55.2 | 86.2 | 9.7 | 95.9 |

(1) xC₂H₆ refers to conversion (per pass) of ethane (%).
(2) sC₂H₄ refers to selectivity towards ethylene (%).
(3) sAA refers to selectivity towards acetic acid (%).
(4) s(C₂H₄ + AA) refers to total selectivity towards ethylene and acetic acid (%).

Surprisingly, it appears from the results in Table 2 that in Exp. 2, wherein accordance with the present invention carbon dioxide was used as a diluent at a relatively high high conversion of ethane (i.e. at least 40%), advantageously the selectivity towards ethylene was substantially higher (87.3%) than that in (comparative) Exp. 1 (84.5%), wherein methane was used instead of carbon dioxide at a similar conversion of ethane (Exp. 1: 51.9%; Exp. 2: 51.5%).

In addition, advantageously the selectivity towards acetic acid was higher in Exp. 2 (8.5%) than that in (comparative) Exp. 1 (8.2%). As discussed above, in a case where ethane is the freshly fed reactant the desired products comprise both ethylene and acetic acid. The total selectivity towards ethylene and acetic acid was advantageously 95.8% in Exp. 2, as compared to only 92.7% in (comparative) Exp. 1.

Also in Exp. 3, wherein carbon dioxide was also used as a diluent, advantageously both the selectivity towards ethylene (86.2%) and the selectivity towards acetic acid (9.7%) were relatively high, at a relatively high conversion of ethane (55.2%). Consequently, in Exp. 3, the total selectivity towards ethylene and acetic acid was also relatively high (95.9%).

We claim:

1. A process of the oxidative dehydrogenation of an alkane containing 2 to 6 carbon atoms and/or the oxidation of an alkene containing 2 to 6 carbon atoms, wherein the alkane and/or alkene is contacted with oxygen in the presence of a catalyst comprising a mixed metal oxide and one or more diluents selected from the group consisting of carbon dioxide, carbon monoxide and steam, and wherein the conversion of the alkane and/or alkene is at least 40%.

2. The process according to claim 1, wherein the conversion of the alkane and/or alkene is of from 45% to 70%.

3. The process according to claim 1, wherein the diluent comprises carbon dioxide.

4. The process according to claim 1, wherein the diluent comprises from 1 to 100 vol. % of carbon dioxide.

5. The process according to claim 1, wherein the alkane is ethane or propane and the alkene is ethylene or propylene.

6. The process according to claim 1, wherein the catalyst is a mixed metal oxide catalyst containing molybdenum, vanadium, niobium and optionally tellurium.
